(19) United States
(12) Patent Application Publication
(10) Pub. No.: US 2025/0170362 A1
(43) Pub. Date: May 29, 2025

(54) Flexible PCB Camera for Bendable Medical Apparatus

(71) Applicant: Canon U.S.A., Inc., Melville, NY (US)

(72) Inventors: Benedict Shia, Needham, MA (US); Tyler Evan Gauvin, Charlton, MA (US); Robert Anthony Zeien, Charlton, MA (US)

(21) Appl. No.: 18/960,794

(22) Filed: Nov. 26, 2024

Related U.S. Application Data
(60) Provisional application No. 63/603,039, filed on Nov. 27, 2023.

(51) Int. Cl. A61M 25/00; A61B 34/20; A61M 25/01

(52) U.S. Cl. CPC A61M 25/00; A61B 2034/2057; A61M 2025/0166

(57) ABSTRACT

A flexible PCB camera and a printed circuit board having an active portion, a flexible portion and a connection portion, wherein the flexible portion is located between the active portion and the connection portion, wherein the flexible PCB camera and the printed circuit board are configured to be inserted into the hollow chamber of an articulated medical device for traversing and capturing images through a hollow core of an apparatus that is capable of large degrees of maneuverability through small spaces of a patient to reach a target and illumination of the target for image sensor capture.

### REFERENCE TO RELATED APPLICATIONS

This application claims priority to U.S. Provisional Application Ser. No. 63/603,039 filed Nov. 27, 2023, the contents of which are incorporated herein in their entirety.

### FIELD OF THE DISCLOSURE

The present disclosure relates generally to apparatus and methods for an articulated medical device having a hollow chamber, and more specifically a flexible PCB camera for insertion into the hollow chamber of the articulated medical device.

### BACKGROUND OF THE DISCLOSURE

Bendable medical instruments such as endoscopic surgical instruments and bendable medical devices are well known and continue to gain acceptance in the medical field. The bendable medical instrument generally includes a flexible body commonly referred to as a sleeve or sheath. One or more tool channels extend along (typically inside) the flexible body to allow access to a target located at a distal end of the body.

By way of example, United States patent publication number 2018/0243900 provides a multi-sectional articulated bendable medical device having bending sections where all sections are comprised of multiple individual nodes/guide rings.

The end objective of the endoscope is advancement into the human body such that the parts inside can be seen, as well as for visualization during navigation. With the use of a camera or videoscope, mages inside the body become accessible and are an invaluable tool for diagnostics, surgery and exploratory viewing. In order to be effective, sufficient lighting must be ensured in order to capture clear images. Therefore, in order to ensure sufficient lighting by illuminating a subject without the blocking of a lens of the camera, a light source part of an endoscopic camera is usually positioned at substantially the same height as the front surface of the lens.

In order to position the light source part at substantially the same height as the front surface of the lens part, a separate printed circuit board (PCB) having the light source part mounted thereon is utilized, and the PCB includes an mage sensor mounted thereon. An example may be represented by United States patent publication number 2011/0118549.

While similar cameras provide the video scope with LED illuminations in the distal camera component with a simple structure, the location of the LED illuminations might need to be more optimal against the imaging sensor. In many cases, the height of the imaging sensors (ex. CMOS sensor) is taller than the LED illuminations, which would cause inefficient illumination with attenuation or degraded imaging quality with stray lights.

As a representative example of this problem, when the imaging sensor (CMOS sensor) and LED illuminations (LED) are mounted on the same surface in a distal camera unit, the height difference between the working surfaces of the CMOS sensor and the LEDs is woefully uneven. Thus the LED's illumination angle (e.g., 140 degrees) will be truncated, resulting in less illumination and generating undesired stray lights.

Furthermore, an exemplary flexible videoscope currently contains a large number of illumination fibers running from the distal tip all the way to the proximal connector. The coaxial cable for the CMOS sensor at the tip runs along with the fibers through the shaft of the flexible videoscope. This construction is very labor and skill intensive, and drives up the cost of the flexible videoscope. The illumination source is a LED in the control box of the flexible videoscope that produces light, which transmits through the illumination fibers and out through the distal end. Since the flexible videoscope is reused due to its cost, the fibers can break as it is continuously used, resulting in light leaks along the shaft and reduced illumination out the distal end.

It is desirable for the flexible videoscope to be a single use, disposable device. In order to reduce the cost of the flexible videoscope, the illumination source(s) is incorporated into the distal tip along with the CMOS sensor. However, the replacement of the illumination fibers with the addition of the light source(s) (LEDs) at the distal end can impact profile such that the flexible videoscope no longer fits inside the tool channel of a catheter.

### SUMMARY

Thus, to address such exemplary needs in the industry, the presently disclosed apparatus teaches a camera module comprising: a flexible printed circuit board having a first arm and a second arm; an image sensor mounted on the first arm; a light-emitting diode mounted on the second arm; and a housing encompassing the flexible printed circuit board, image sensor and light-emitting diode, wherein both the first arm and second arm are manipulated, independent of each other, to align the image sensor with a light emitting element of the light-emitting diode.

In various embodiments, the orientation of the aligning of the image sensor with the light emitting element is perpendicular to the length of the flexible printed circuit board.

In other embodiment, the aligning of the image sensor with the light emitting element comprises bending each of the image sensor and light-emitting diode by approximately ninety degrees.

In further embodiments, the image sensor is a camera.

If it further contemplated that the aligning of the image sensor with the light emitting element results in a top surface of the image sensor and a top surface of the light emitting diode being parallel with each other.

In yet another embodiment, the camera module further comprises a third arm configured for manipulation to align with the image sensor and the light-emitting diode. In addition, a second light-emitting diode is mounted to the third arm.

It is further disclosed herein that the first arm or second arm may be angled towards the center of the camera module.

The subject innovation also teaches the image sensor and the light-emitting diode mounted on the same side of the flexible printed circuit board.

It is also contemplated that the first arm or second arm may be folded multiple times.

In additional embodiment, a conductor of a coaxial cable is soldered to the flexible PCB at the proximal end of the flexible PCB.

Furthermore, the solder connection of the coaxial cable may be disposed on one surface of the flexible PCB.

In other embodiments, the solder connection of the coaxial cable is disposed on both surfaces of the flexible PCB.

An additional embodiment provides a printed circuit board having an active portion, a flexible portion and a connection portion, wherein the flexible portion is located between the active portion and the connection portion, wherein the active portion has electrically attached thereto at least one image sensor, the flexible portion comprises a flexible PCB material having printed thereon conductors electrically connecting the image sensor in the active portion and the connection portion, and the connection portion comprises means for electrical connection to a power and/or control source; wherein no attachment means or soldering is present in the flexible portion.

Furthermore, in an additional embodiment there is provided a camera module comprising the printed circuit board having an active portion. a flexible portion and a connection portion. wherein the flexible portion is located between the active portion and the connection portion, wherein the active portion comprises at least a first arm and a second arm, the image sensor mounted on the first arm, a light source mounted on the second arm; and wherein the flexible portion is configured to extend a distance lengthwise through an endoscope for a distance that is at least five times the diameter of the endoscope.

The present disclosure further teaches a method for employing a camera module comprising: a flexible printed circuit board having a first arm and a second arm; an image sensor mounted on the first arm, a light-emitting diode mounted on the second arm; and a housing encompassing the flexible printed circuit board, image sensor and light-emitting diode, wherein both the first arm and second arm are manipulated, independent of each other, to align the image sensor with a light emitting element of the light-emitting diode: wherein the method incorporated use of the camera module in a medical setting.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures which illustrate various embodiments, objects, features, and advantages of the present disclosure.

FIG. 1 is a block diagram of an exemplary bendable medical device

incorporating various ancillary components, according to one or more embodiment of the subject apparatus, method or system.

FIG. 2 provides a cut-away perspective view of an exemplary bendable medical device, according to one or more embodiment of the subject apparatus, method or system.

FIG. 3 is a cut-away perspective view of an exemplary bendable medical device, according to one or more embodiment of the subject apparatus, method or system.

FIG. 4A-4D provide a side profile (4A) and front cut-away (4B-4D) views of an exemplary bendable medical device, according to one or more embodiment of the subject apparatus, method or system.

FIG. 5 depicts a side of view of the LED light projection, according to one or more embodiment of the subject apparatus, method or system.

FIG. 6A provides a perspective view of a flexible PCB Camera, according to one or more embodiment of the subject apparatus, method or system. FIG. 6B provides a perspective view of a flexible PCB Camera which has been bent into a final folded position, according to one or more embodiment of the subject apparatus, method or system. FIG. 6C provides a perspective view of a flexible PCB Camera, according to one or more embodiment of the subject apparatus, method or system.

FIG. 7 depicts a side view of an exemplary flexible PCB Camera within a housing, according to one or more embodiment of the subject apparatus, method or system.

FIG. 8 depicts a front view of an exemplary flexible PCB Camera, according to one or more embodiment of the subject apparatus, method or system.

FIG. 9 provide a top profile of a flexible PCB Camera, according to one or more embodiment of the subject apparatus, method or system.

FIG. 10 provide a top profile of a flexible PCB Camera, according to one or more embodiment of the subject apparatus, method or system.

FIG. 11 provides a top profile of a flexible PCB Camera, according to one or more embodiment of the subject apparatus, method or system.

FIGS. 12A and 12B provide a top and side perspective view, respectively, of a flexible PCB Camera, according to one or more embodiment of the subject apparatus, method or system. FIGS. 12C, 12D and 12E provide top and perspective views, respectively, of exemplary PCB layouts in accordance with one or more elements of the subject disclosure. FIG. 14 provide a top profile of an exemplary PCB layout in a serpentine pattern in accordance with one or more elements of the subject disclosure.

FIGS. 13A and 13B provide a top profile of a flexible PCB Camera, according to one or more embodiment of the subject apparatus, method or system.

FIG. 14 provides a top perspective view of an exemplary PCB layout in accordance with one or more elements of the subject disclosure.

FIG. 15 depicts an expanded top view of an exemplary PCB layout in accordance with one or more elements of the subject disclosure.

FIG. 16 provides a top profile of an exemplary PCB layout in a serpentine pattern in accordance with one or more elements of the subject disclosure.

FIG. 17 depicts an expanded top view of an exemplary PCB layout in accordance with one or more elements of the subject disclosure.

FIG. 18 provides a photograph of a device according to one or more elements of the subject disclosure.

Throughout the Figures, the same reference numerals and characters, unless otherwise stated, are used to denote like features, elements, components or portions of the illustrated embodiments. In addition, reference numeral(s) including by the designation “ ’ “(e.g. 101′ or 24′) signify intermediary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

The subject innovation will now be relayed in detail through the description of the exemplary figures, commencing with the general system associated with the bendable medical device.

### DETAILED DESCRIPTION

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

### Catheter System

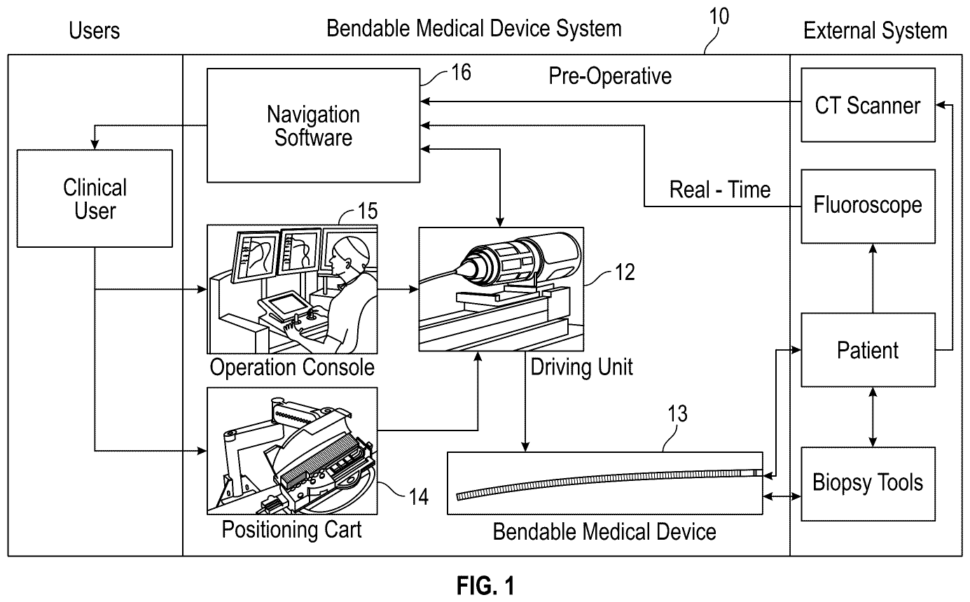

FIG. 1 is a system block diagram of an exemplary bendable medical device system 10 incorporating various ancillary components intended to amass a complete medical system. The bendable medical device system 10 comprises a driving unit 12, a bendable medical device 13, a positioning cart 14, an operation console 15 and navigation software 16. The exemplary bendable medical device system 10 is capable of interacting with external system component and clinical users to facilitate use in a patient.

The navigation software 16 and the driving unit 12 are communicatively-coupled via a bus to transmit/receive data between each other. Moreover, the navigation software 16 is connected and may communicate with a CT scanner, a fluoroscope and an image server (not in Figures), which are ancillary components of the bendable medical device system 10. The image server may include, but is not limited to, a DICOM™ server connected to a medical imaging device including but not limited to a CT and/or MRI scanner and a fluoroscope. The navigation software 16 processes data provided by the driving unit 12 and data provided by images stored on the image server, and/or images from the CT scanner and the fluoroscope in order to display images onto the image display.

The images from the CT scanner may be pre-operatively provided to navigation software 16. With navigation software, a clinical user creates an anatomical computer model from the images. In this particular embodiment, the anatomy is that of a lung with associated airways. From the chest images of the CT scanner, the clinical user can segment the lung airways for clinical treatments, such as biopsy. After generating the lung airway map, the user can also create plan to access the lesion for the biopsy. The plan includes the airways to insert and maneuver the bendable medical device 13 leading to the intended target, which in this example is a lesion.

The driving unit 12 comprises actuators and a control circuitry. The control circuitry is communicatively-coupled with operation console 15. The driving unit 12 is connected to the bendable medical device 13 so that the actuators in the driving unit 12 operate the bendable medical device 13. Therefore, a clinical user can control the bendable medical device 13 via the driving unit 12. The driving unit 12 may also be physically connected to a positioning cart 14. The positioning cart 14 includes a positioning arm, and locates the driving unit 12 and the bendable medical device 13 in the intended position with respect to the target/patient. The clinical user can insert, maneuver and retreat the bendable medical device 13 to perform medical procedures, here a biopsy in the lungs of the patient.

The bendable medical device 13 can be navigated to the target in a patient based on the plan by the clinical user's operation. The bendable medical device 13 includes a tool channel 108 for various tools (e.g. a biopsy tool). The bendable medical device 13 can guide the tool to the lesion of the patient. In one example, the clinical user can take a biopsy sample from the lesion with a biopsy tool.

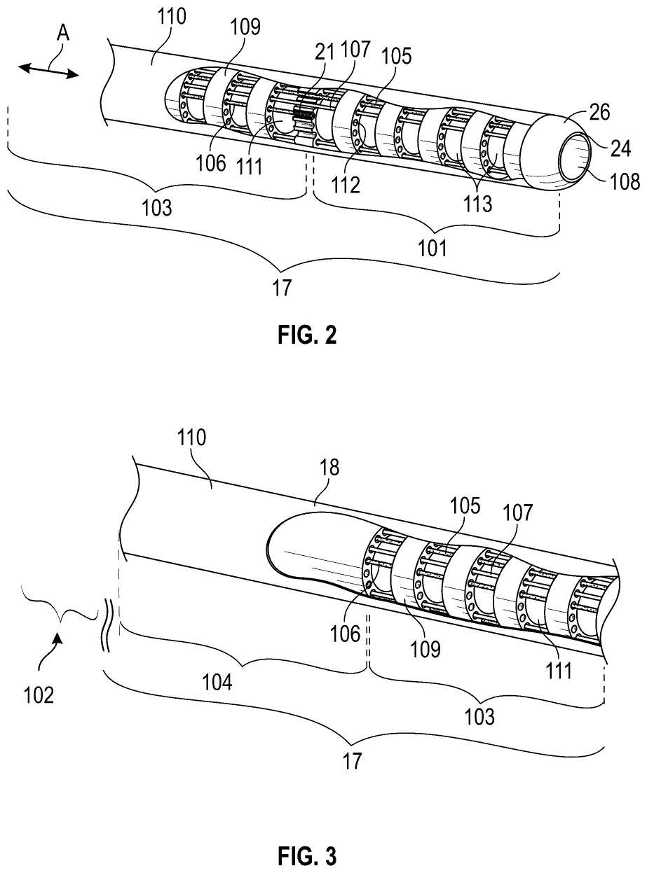

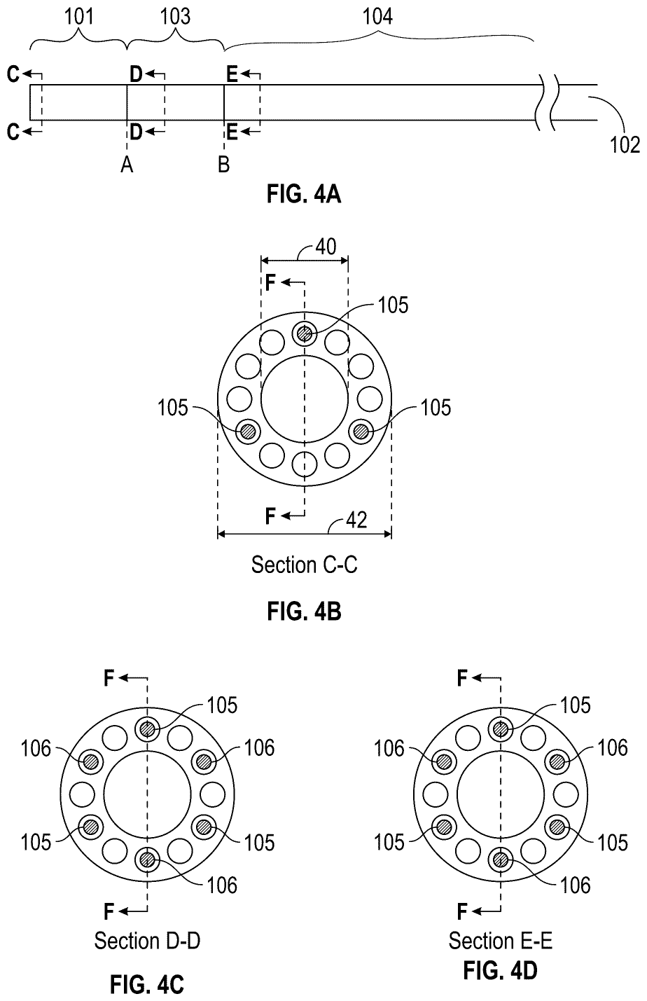

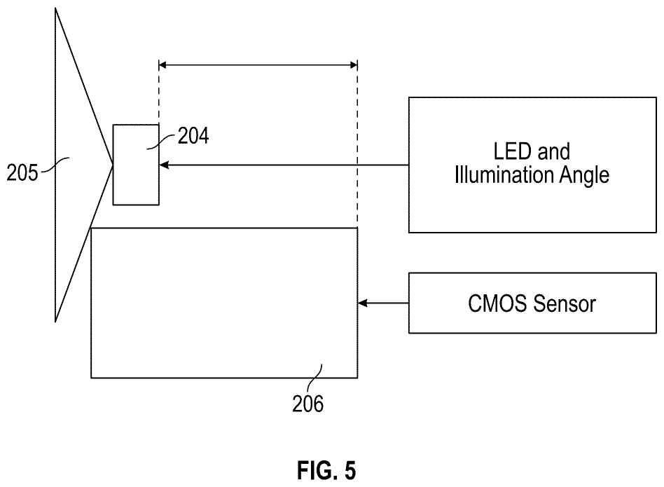

As depicted in FIGS. 2 and 3, the distal section 101 of the bendable medical device 13 comprises multiple guide rings 109, wherein the guide rings 109 are configured a distance apart from one another and do not contact one another. The guide rings 109 are held in place by the cylindrical wall 18, which comprises an inner lining 111 and an outer lining 110, which provides bendable support to the bendable body 17 while retaining the guide rings 109 in a constant position along the axial direction of the bendable body 17. The inner lining 111 creates an inner diameter 40 (as shown in FIG. 4B) and the outer lining 110 creates an outer diameter 42 (as shown in FIG. 4B), wherein the inner diameter 40 establishes the tool channel 108. The edge of the bendable body 17 may be rounded by an atraumatic tip 26, to further diminish any harm to the internal elements of a patient as the bendable body 17 is advanced.

The adjacent guide rings 109, are attached to the inner lining 111 and/or outer lining 110, with cavities 113 created between the adjacent guide rings 109, distributed along the longitudinal direction of the bendable body 17. Each guide ring 109 contains at least two guide holes 112, extending the length of the guide ring 109 parallel with the length of the bendable body 17, for slideable housing of the driving wires 105-107. Furthermore, each guide hole 112 within the guide rings 109, is configured to accept an anchor 21, which is displaced at the end of the driving wires 105-107, to be embedded into the guide ring 109. In FIG. 2, the proximal driving wire 106 depicts the anchor 21, configured at the distal end of the intermediate bendable section 103. The space between adjacent guide rings 109, in cooperation with the resilient inner lining 111 and outer lining 110, allows the bendable body 17 to achieve a greater range of bending motion due to the open space between the guide rings 109, without kinking.

The tool channel 108 is configured to extend the length of the bendable body 17, wherein the proximal end of the bendable body 17 provides access to clinical users for inserting/retreating a medical tool. For example, a clinical user can insert and retrieve a biopsy tool through the tool channel 108 at the proximal end of the bendable medical device 13, which tool is then used at the distal end of the bendable medical device 13.

FIGS. 4B through 4D depict cross-sectional views of exemplary bendable medical devices shown in FIG. 4A. FIG. 4B depict the cross-section view at the “C” line in FIG. 4A, while FIG. 4C depicts the cross-sectional view at the “D” line, while FIG. 4D shows us the cross-sectional view at the “E” line of FIG. 4A.

As seen in the cross-sectional views in FIG. 4A-4D, the bendable body 17 includes a set of distal driving wires 105, a set of intermediate driving wires 106, and a set of proximal driving wires 107, housed in the bendable body 17, wherein each of the set of driving wires 105, 106 and 107, corresponds to the distal, intermediate and proximal bendable sections 101, 103 and 104, respectively. The inner lining 111 creates the inner diameter 40 of the wall and establishes the tool channel 108, while the outer lining 110 creates the outer diameter 42 of the bendable body 17.

The bendable body 17 houses each of the driving wires 105-107 in corresponding guide holes 112, configured along the longitudinal direction of the bendable body 17. The guide holes 112 allow for slideable movement of the driving wires 105-107 along an axial direction of the bendable body 17. The driving wires 105-107 are terminated at the distal end of each bendable section 101, 103 and 104. The distal driving wires 105 are terminated at the distal end of the distal section 101 with anchors 21, and are configured apart from each other by approximately 120 degrees within the bendable body 17. The distal driving wires 105 are connected to the driving unit 12 at the proximal end of the wires 105. The driving unit 12 induces pushing or pulling forces to move the distal driving wires 105 by actuating those wires, and bends the bendable body 17 at the distal bendable section 101. The intermediate and proximal driving wires 106 and 107 are similarly configured for their corresponding bendable sections 103 and 104, respectively.

Accordingly, by pushing and pulling the driving wires 105 through 107 the proximal, intermediate and the distal bendable sections 104, 103, and 101, respectively, can individually bend the bendable medical device 13, in all three dimensions.

### Flexible PCB

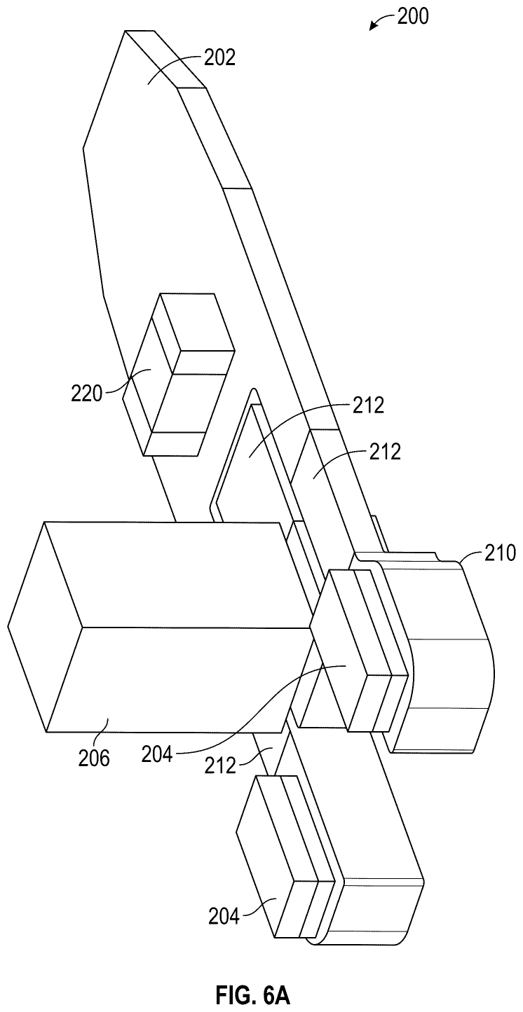

FIG. 5 et seq. depict the subject innovation, namely the flexible PCB camera 200. While the flexible PCB camera 200 may be described herein as utilizing LEDs and a CMOS sensor, these components can more generally be identified as one or more lighting sources, and an imaging sensor. Such lighting sources may be LEDs, which will be used herein as an exemplary embodiment, but could also be one or more other types of lighting sources which would be of appropriate size, shape, and type, which can be determined by one of skill in the art. Similarly, an imaging sensor can more generally be a camera or other sensor, including, but not limited to, a CMOS sensor which will be used herein as an exemplary embodiment, but which could also be one or more other types of imaging sensors, the suitability of which can be determined by one of skill in the art. Such imaging sensor can be used to record stationary images, videos or both.

The flexible PCB camera 200 incorporates a novel flexible printed circuit board 202 that can realize the advantageous layout in FIG. 5, wherein the lighting sources 204 working surface should be at a similar level to the working surface of the image sensor 206, providing an illumination angle 205 that provides illumination directly in front of the image sensor 206, thus preventing attenuation and stray light caused by relative height differences. In exemplary embodiments, more than one lighting source 204 can be used such that the collective illumination angles 205 overlap to provide illumination in front of the image sensor 206. As stated, the image sensor 206 can be a camera, CMOS sensor or other appropriate device, and the lighting source 204 can be an LED or other suitable lighting element.

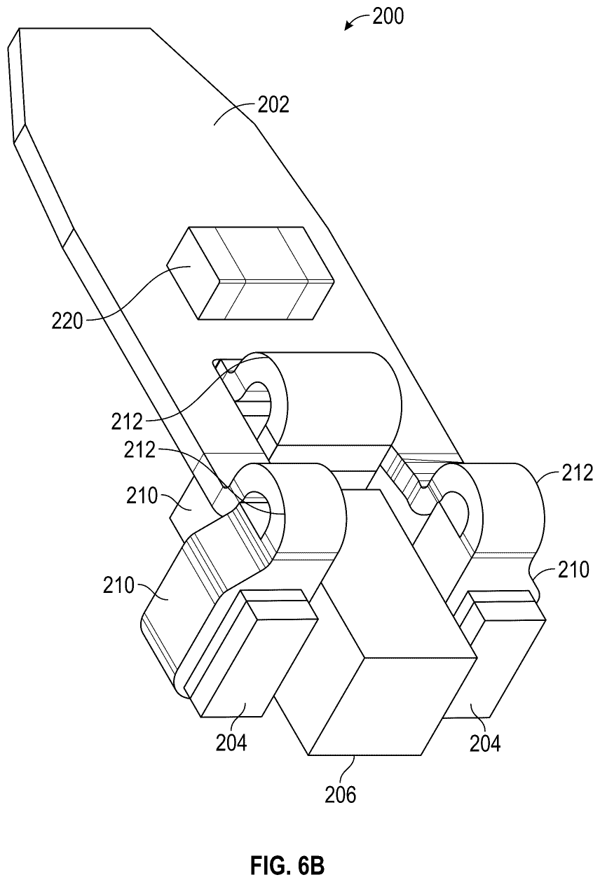

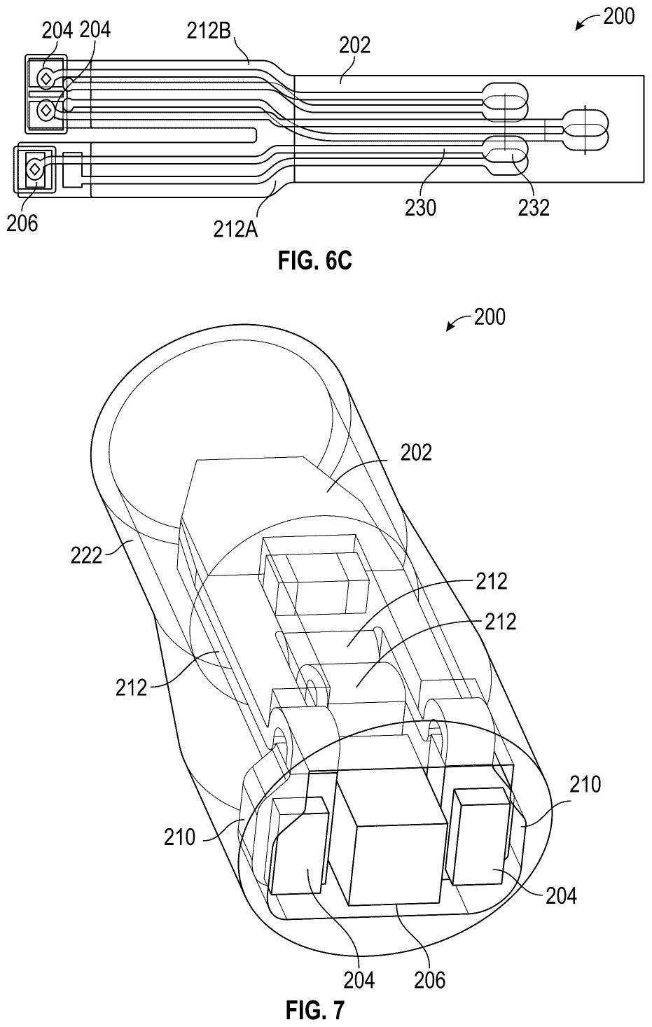

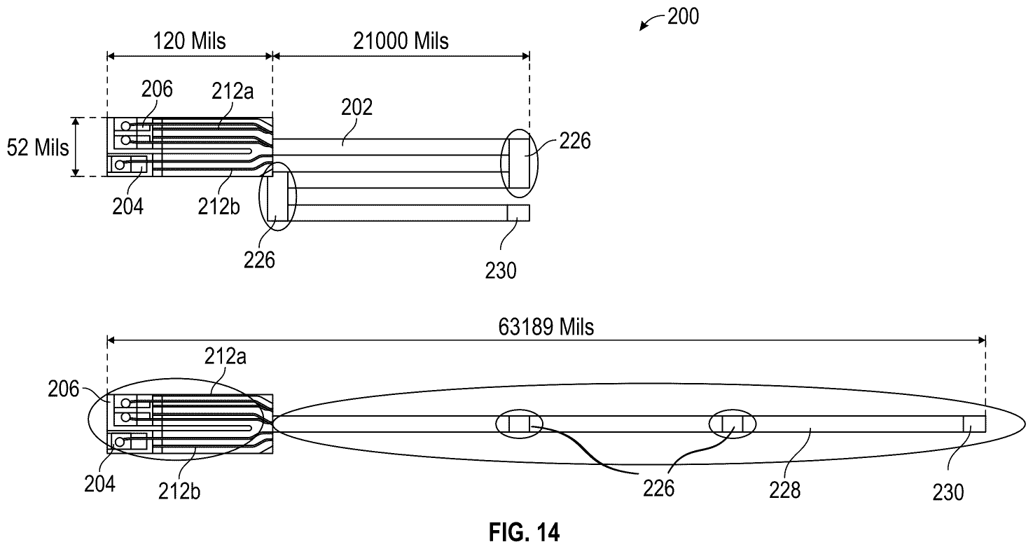

FIG. 6A-B provides further detail of an exemplary device that can be used to achieve such an advantageous layout, wherein a thin, flexible printed circuit board (“PCB”) 202 can be used to mount the various components 208 (including image sensor 206, lighting source(s) 204, and optional resistor 202) of the flexible PCB camera 200. The flexible PCB can have a single coaxial cable 234 (not shown) connected to the PCB 202 and its components. The coaxial cable 234 can be soldered on one or both sides of the PCB 202. Alternatively, the flexible PCB 202 could be formed into a long, thin ribbon cable 228 (as shown in FIGS. 6C, 14, and 15), or it could be formed into a rectangular shape with the conductors 224 shaped into a serpentine pattern, which serpentine pattern can then be unfolded to increase the length of the flexible PCB 202 (see FIG. 16). Because the components are rigid but the PCB 202 is not, the PCB could flex away from the components, compromising the connections. Additional support structures 210 (further described in FIGS. 6B and 7) can be added to the locations where the image sensor 206 and image sensor(s) 204 are mounted for better stability and connection.

As shown in FIG. 6A-B, all the components 208 are configured on the same side of the flexible PCB 202. This simplifies the structure and manufacturing of the PCB 202, as there is no need for conductors to be on both sides of the PCB 202, making the PCB 202 thinner as well as easier to assemble and service, if necessary.

As seen in FIG. 6A, the components 208 are attached to the PCB 202 while the PCB 202 is flat. The PCB 202 may be trimmed with various length arms 212 for each individual component 208 (CMOS Sensor and LED's) at the desired length and configuration. The flexible PCB 202 can then be manipulated to better align the image sensor 206 and the image sensor(s) 204, such that the optimal similarity in working levels is achieved. Because the arms 212 are capable of having different lengths, they can be flexed at different points to bring the image sensor 206 and image sensor(s) 204 closer to the same height, when the respective portions of the PCB 202 is bent.

FIG. 6B depicts the same PCB 202 and components 208, as seen in FIG. 6A, however, the respective portions of the PCB 202 are bent, and the length of the arms 212 keep the components 208 at their desired positions. As a result, the portion of the PCB 202 housing the two image sensor(s) 204 are bent at approximately 90 degrees and the arms 212 hold the image sensor(s) 204 in position. Similarly, the portion of the PCB 202 housing the image sensor 206 is bent at approximately 90 degrees and the arm 212 holds the image sensor 206 in position. As the height of the image sensor 206 is greater than the height of the image sensor(s) 204, the arm 212 for the image sensor 206 is trimmed further back on the PCB 202 to accomplish the desired alignment of the forward facing portion of the image sensor(s) 204 and image sensor 206 when the arms 212 are bent or flexed into their final position. The exact configuration of the PCB 202, components 208 and arms 212 can be made depending on the dimensions of the selected components 208, and their desired alignment with respect to each other. For example, the configuration of the PCP 202, components 208 and arms 212 can be determined to provide an alignment of the image sensor(s) 204 and image sensor 206 as shown in FIG. 5. FIG. 6B and FIG. 7 both depict an exemplary positioning of additional support structures 210, which can be added to arms 212 in order to provide additional support for the LEDs 204 and image sensor 206.

Depending on the length of the arms 212 and how far the arm needs to be pulled back in order to align the components 208, the arm 212 can undergo a series of angular bends, for instance from 0 to 90 degrees or more, or the arm 212 can be bent or flexed in a series of waves or undulations, any of which can be used provided they result in the component 208 being in the appropriate alignment.

The flexible PCB 202 can be made of materials including, but not limited to PI (polyimide) film, PET (polyester) film, and polymer films such as PEN (polyethylene nphthalate), PTFE and aramids, etc., as would be understood by one of skill in the art. Bending or flexing of the arms 212 can be accomplished mechanically, including via automated means. Once bent or flexed, the arms 212 may be configured to maintain their desired position. If desired, the arms 212 can be affixed into position by any means standard in the field, including, but not limited to the use of adhesives, fasteners, or a state or temperature change of the material, as would be understood by those of skill in the art.

As depicted in FIG. 7, the flexible PCB camera 200 therefore is comprised of a housing 222, in which is contained the flexible PCB 202, having all of the components 208 thereon, including image sensor 206, lighting source(s) 204, optional resistor 220, which flexible PCB 202 has been folded or bent into its final configuration, thereby allowing the image sensor 206 and lighting source(s) 204 to be in optimal alignment for visualization when directed through a bendable medical device 13. The housing can be made, for instance, by 3D-printing, or by other means. The housing material can be a thermoplastic polymer, such as acrylonitrile butadiene styrene (ABS), an ABS-like material, or similar. The flexible PCB camera 200 is additionally electrically connected to a controller and a power source via a connection means that is sufficiently long to allow the flexible PCB camera 200 to travel the distances required by the bendable medical device 13 as it is inserted into a patient.

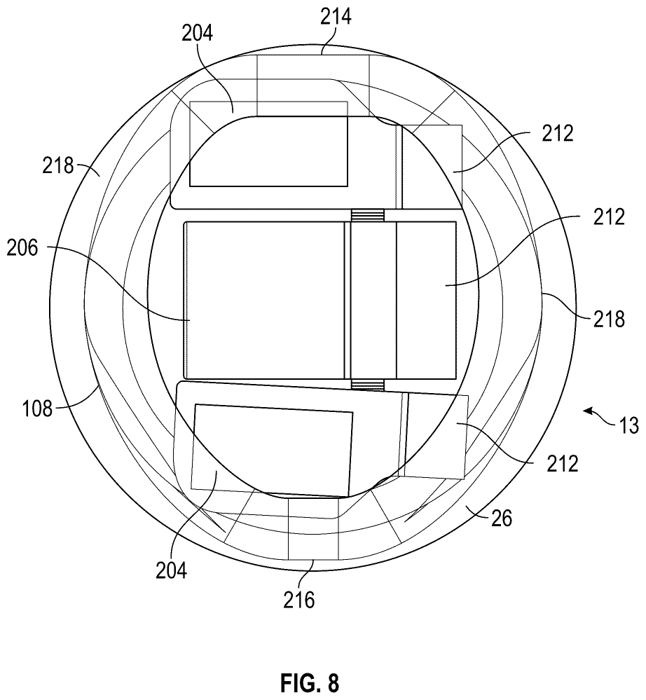

The desired alignment of the components (lighting source(s) 204 and image sensor 206) on the PCB 202, results in the conformed PCB seen in FIG. 8, having a compacted diameter, and therefore suitable for entry into the tool channel 108 of the bendable medical device 13.

As seen in FIG. 8, the distal housing of the tool channel may be designed with a trapezoidal shape with an upper edge 214, a lower edge 216, and curved sides 218. This trapezoidal shape represents a smaller cross sectional area than a standard circular shape, and the small profile of the PCB 202 can fit into the trapezoidal shape. Design of distal housing can incorporate further internal features to support the flexible PCB 202.

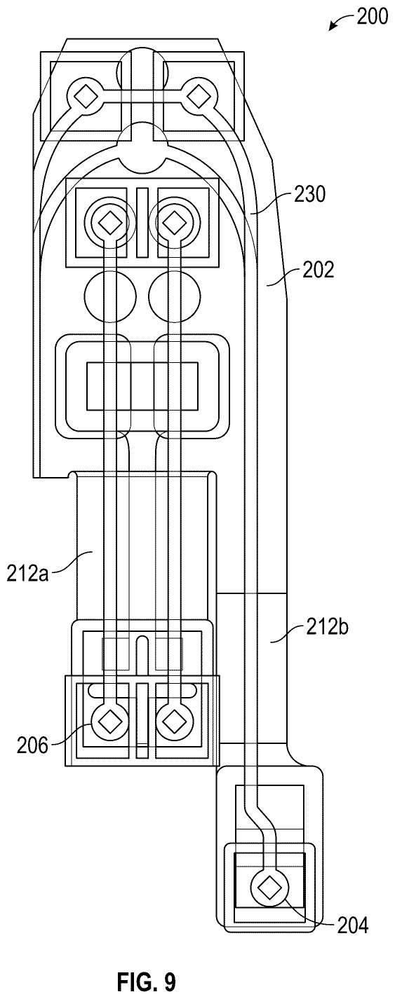

FIGS. 9, 10 and 11 provide various arrangements for the PCB 202, having different lengths or number of arms 212, as well as different angles and bends in the arms 212a, 212b, and 212c. As can be appreciated, the examples provided here are not limited and can be arranged in different configurations to accommodate the components involved and lengths needed for proper use and alignment of the components. By way of example, FIG. 9 depicts a flexible PCB 202 having two arms 212, wherein the first arm 212a has an image sensor 206 at distal end, and the second arm 212b has a lighting source 204 mounted on the distal end.

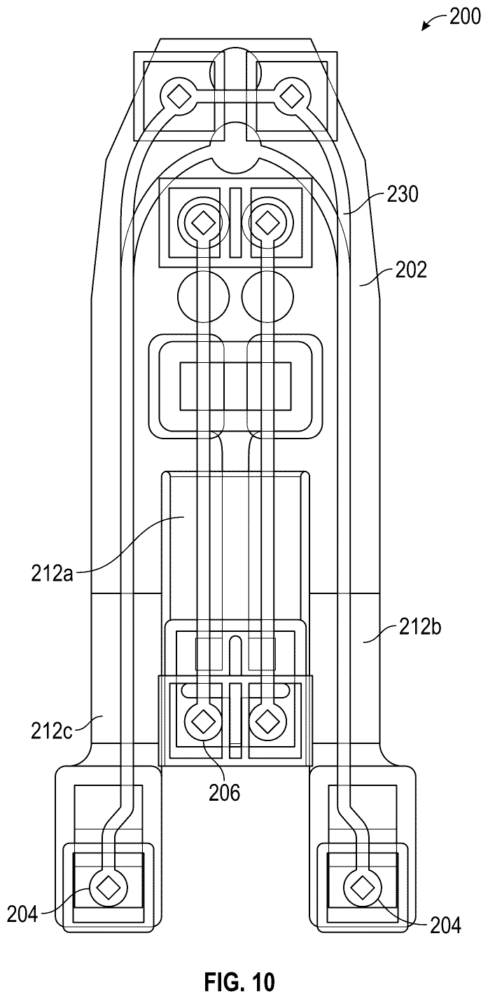

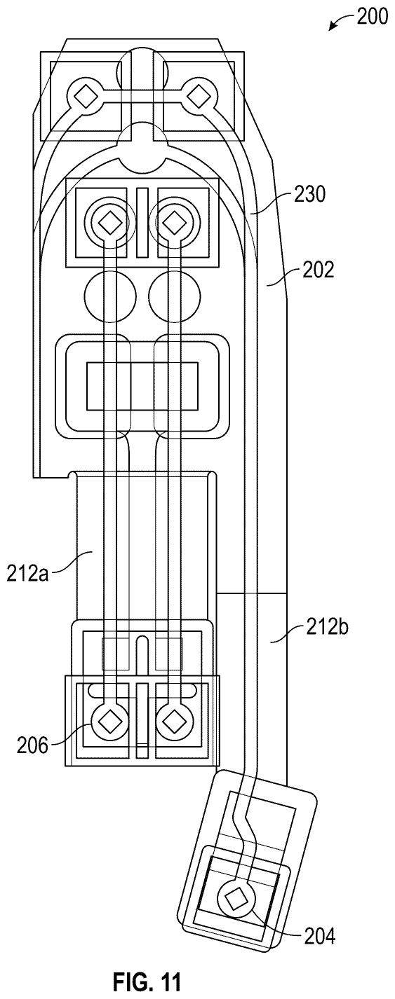

FIG. 10 depicts a flexible PCB 202 having three arms 212, wherein the first arm 212a has an image sensor 206 at distal end, the second arm 212b has a lighting source 204 mounted on the distal end, and the third arm 212c has a second lighting source 204 mounted on the distal end. FIG. 11 provides the same arrangement as seen in FIG. 9, however, the second arm 212b has a slight bend at the distal end of the arm 212b to better position the LED to provide lighting. Thus, using FIGS. 9-11 as exemplary embodiments, one of skill in the art can determine the appropriate number of arms 212, their lengths, and any additional configuration details, such as bends or curvature.

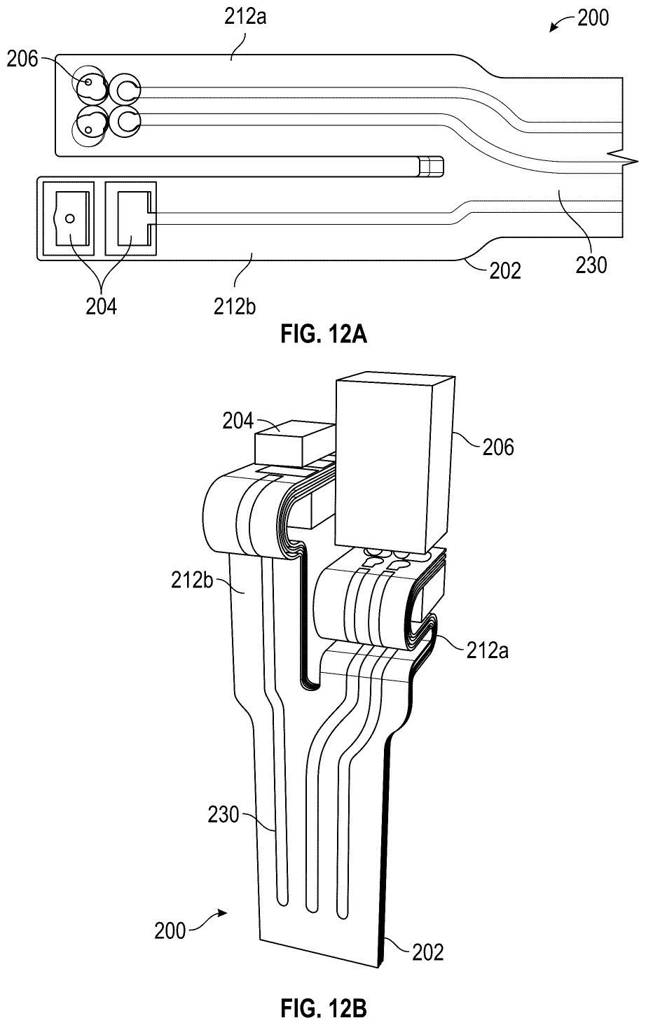

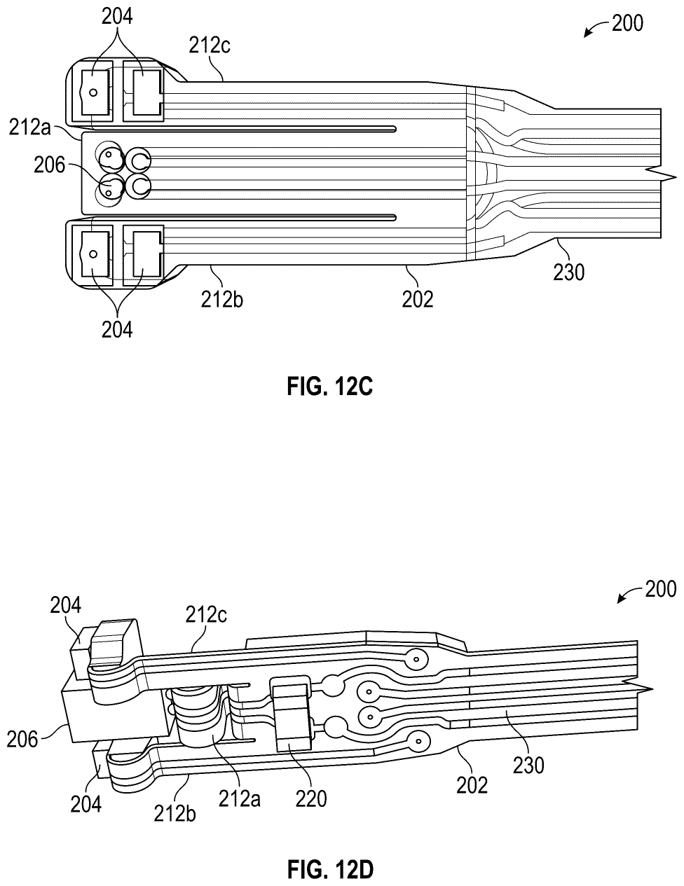

As can be seen in FIGS. 12A, 12D,, 13A and 13B, one or more of arms 212a, 212b, 212c may have multiple bends. As shown in FIG. 12A, 12C and 13A, in order to simplify design and/or manufacturing, the arms 212 may be cut to fairly equal lengths, so the arm 212 with the image sensor 206, which is the taller part, must be folded more times to balance out the height of the image sensor with the arm 212 having the shorter lighting source 204. Arm 212a having multiple bends to align the image sensor 206 with lighting source 204 is depicted in FIGS. 12B, 12D, and FIG. 13B, although the manner in which the folds or bends are created is different. FIG. 12B and 12d provide bends in arm 212a that have more curvature to them, whereas FIG. 13B depicts arm 212A as having folds or bends that are more geometric or angular, such as a series of approximately 90 degree folds or bends. In another configuration, arms 212a, 212b and/or 212c can have different lengths, so that a simple fold for each arm/component would balance the heights. Furthermore, as seen in FIG. 12A, multiple lighting sources 204 may be mounted on a single arm 212b, thus eliminating the need for a second arm having a lighting source. Put another way, the lengths of the arms are calculated so that a single bend or multiple bends is sufficient to level the height of the lighting source(s) 204 to the height of the image sensor 206. Such bends can be configured as necessary to achieve the desired length, including one or more bends of 0 to 179 degrees, including, for instance, 90 degrees. In this manner, the aligning of the image sensor 206 with the light source 204 is oriented perpendicular to the length of the flexible printed circuit board.

More specifically, FIGS. 12C and 12D depict a flexible PCB 202 having three arms 212, wherein the first arm 212a has an image sensor 206 at the distal end, the second arm 212b has a light source 204 mounted on the distal end, and the third arm 212c has a second light source 204 mounted on the distal end. For more balance and increased illumination, the flexible PCB 202 could have 3 arms 212, wherein the image sensor 206 would be soldered to the middle arm 212a, and one or more lighting source(s) 204 would be soldered to each of the other arms 212b and 212c. As an advantageous byproduct, having multiple LED's, each LED can be supplied with less power, resulting in lower heat generation.

In yet another alternative embodiment, as provided in FIG. 12E, if a 3D image is desired, two or more image sensors 206 may be used with a single light source 204 on the three arm configuration. Here a single light source 204 would be soldered to the middle arm 212a and two image sensors 206 would be mounted or soldered onto the other two outer arms 212b and 212c.

### Ribbon Cable

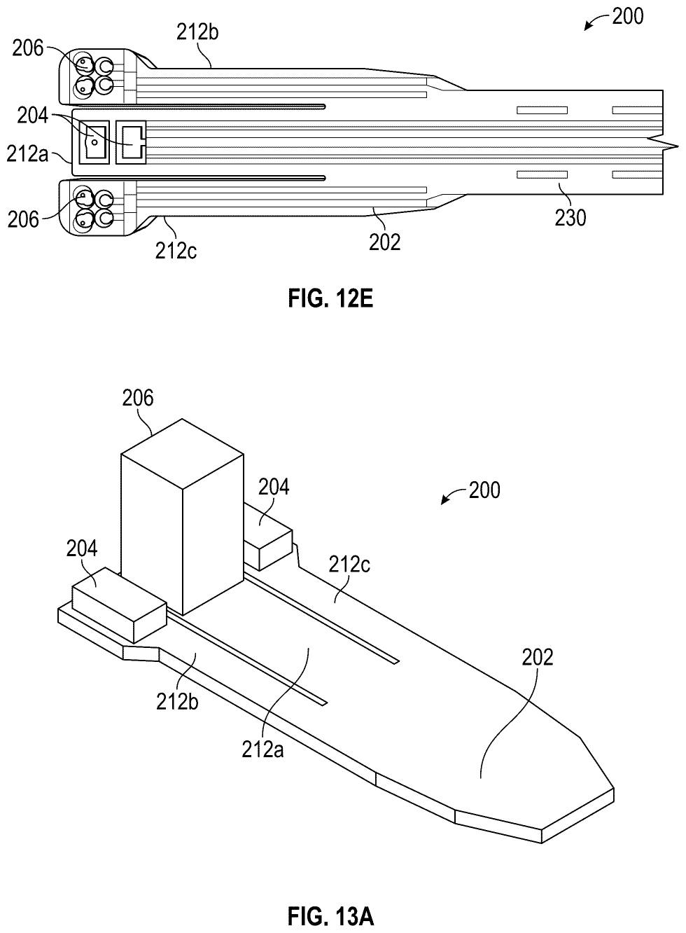

As shown in FIG. 14, the flexible PCB 202 as previously described can be lengthened, such that the distance between components 208 and an electrical connection means 230 is elongated. In this manner, the extended flexible PCB 202 include a thin flexible ribbon cable 228 extended between the initial, active portion of the PCB 202 containing components 208, and a connection portion 238 which comprises conductors 224 to which an external power source and/or controller can be attached. The extension of the flexible PCB 202 into ribbon cable 228 allows for all soldered electrical connections to be located at the connection portion 238, allowing the flexible portion of the ribbon cable 228 to be free of any soldering or other connection points, such that the ribbon cable 208 will be fed through a camera shaft, such that the camera and camera shaft will be more easily maneuverable through the tool channel 108 of the bendable medical device 13.

As shown in FIGS. 14 and 16, the electrical traces 224 embedded in the flexible PCB 202 material can run the whole length of the ribbon cable 228 and will extend straight, or follow the serpentine pattern cut out of the rectangular PCB 202 material. The serpentine ribbon cable 228 could be pulled and folded or twisted at the fold areas 226 to straighten out the ribbon cable 228. In these embodiments, the connection means 230 is located at the proximal end of the connection portion 238 and the electrical connection means 230 can be a ZIP connector or similar, which can then be attached to a coaxial cable 234 or other power source and/or controller.

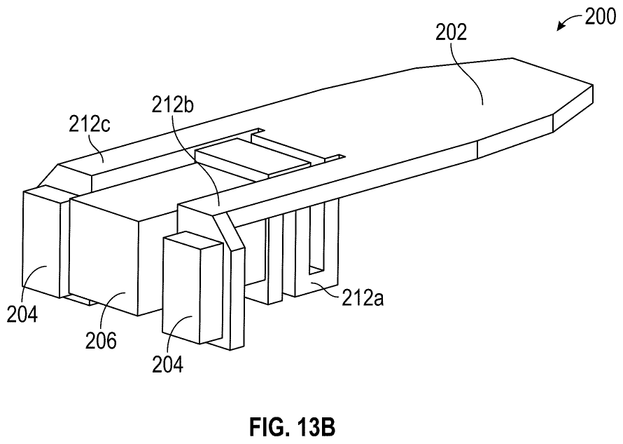

The alteration of the PCB 202 from the configuration shown in FIGS. 6 to 13 to that of having an integrated ribbon cable 228 can result in increasing the length of the PCB 202 from around 5 mm to up to around 1.5m in total length. For example, in a PCB 202 having a length of up to around 1.5m, the flexible printed circuit board 202 could extend the full length of the shaft extrusion, configured as a ribbon cable 228, as shown in FIG. 15. If the PCB 202 was cut having a serpentine section as seen in FIGS. 14 and 16, the serpentine section can be form the ribbon cable 228 that could be pulled straight to fit through a tool channel 108.

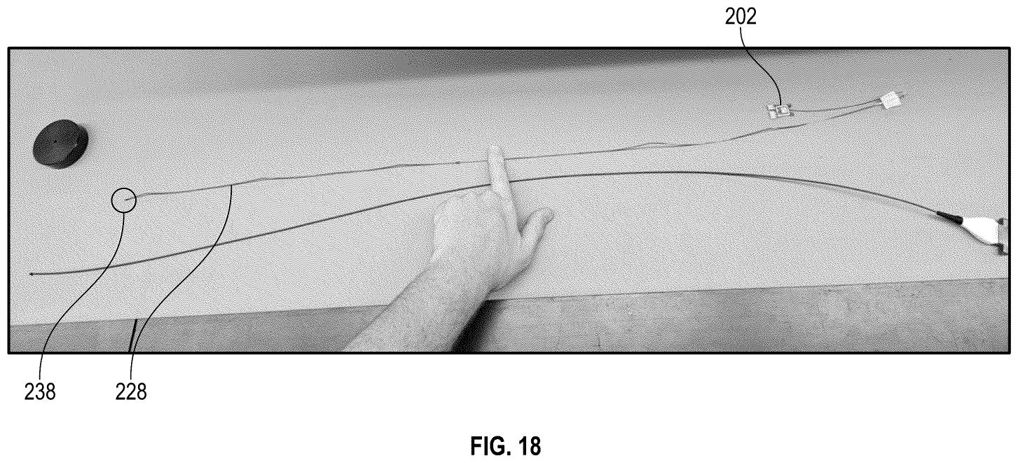

For instance, an exemplary printed circuit board 202 can be about 1.5 meters in length with a thickness of approximately 0.005 inches and a width of 0.040 inches. A photograph of an exemplary device is found in FIG. 18. The exceptional flexibility of the printed circuit board 202 allows for easy advancement and retraction through the most tortuous of pathways created by the bendable medical device 13. Additionally, the flexibility on the printed circuit board 202 add negligible tension to the curvature of the printed circuit board 202, thus allowing for easy advancement and retraction of the printed circuit board 202 without effecting the shape of the bendable medical device 13.

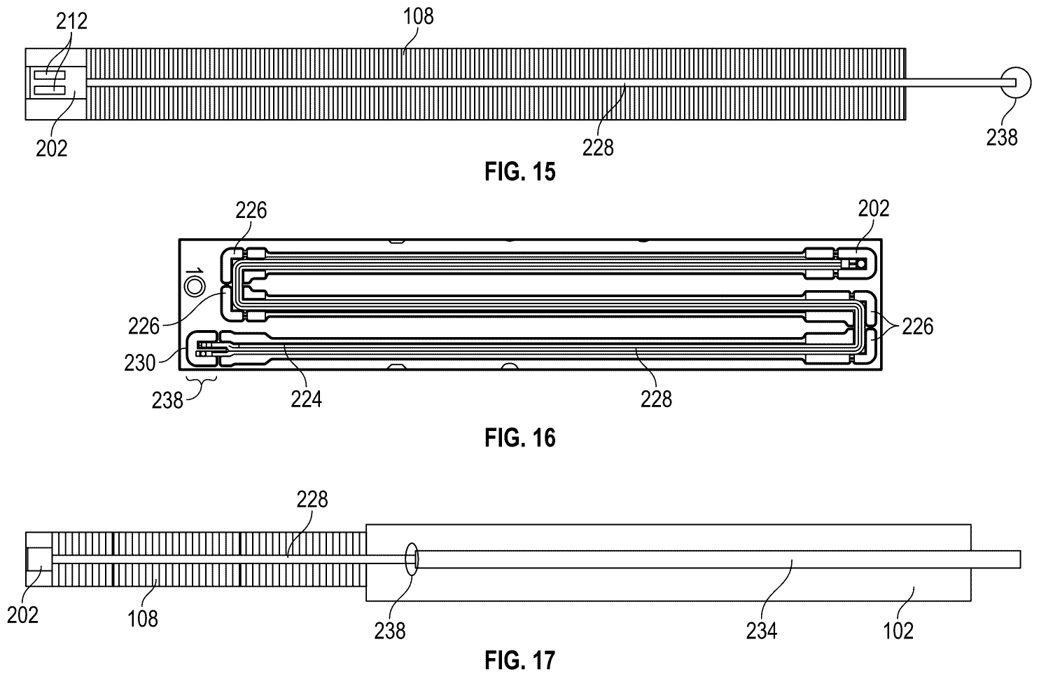

In other embodiments, it may be beneficial for the ribbon cable to be a length such that it extends a distance lengthwise through an endoscope for a distance that is at least five times the diameter of the endoscope. In such an embodiment, the length of the ribbon cable 228 would extend through the bendable portions (101, 103 and 104) of bendable medical device 13, allowing for increased flexibility and maneuverability of the PCB camera 200 within the tool channel 108, while an attached coaxial cable 234 would traverse the non-bendable, proximal portion 102 of the bendable medical device 13. Such an embodiment is shown in FIG. 17.

FIG. 17 provides the same arrangement as seen in FIG. 15, however, instead of the ribbon cable 218 extending all the way through the shaft extrusion, it could extend far enough back to be proximal to the distal bending section of the bendable medical device 13, conserving the amount of flexible board material needed. As rigid length is not as critical in this section of the bendable medical device 13, more of the camera 206 working length would be taken up by a coaxial cable 234, which would reduce the cost of the camera 206.

The present disclosure provides multiple advantages over the existing art, including, a reduced cost in manufacturing the flexible PCB camera 200, wherein the manufacture of the PCB 202 can be automated and loading a single coaxial cable or a PCB 202 having a ribbon cable 228 through the camera shaft is less labor intensive than loading the cable and a multitude of illumination fibers.

Further advantages of the present disclosure include the elimination of a frame to support the flexible PCB 202, or to which the flexible PCB 202 is attached. The concept detailed herein has additional support structures 210 attached to the flexible PCB 202 on the opposite side to of the PCB 202 to where the image sensor 206 and light source 204 are attached. This keeps the overall structure smaller in mass and profile, and further declutters the PCB Camera 200. The smaller mass and profile is also possible because the manner in which the flexible PCB 202 is conformed allows all the components 208 to be soldered to the same side of the PCB 202. This additionally simplifies the structure and manufacturing of the PCB 202. Further, the conformation of the flexible PCB 202 keeps the light sources 204 in line with the image sensor 206, both axially and at similar heights. This allows better and more even illumination for the image sensor 206. In one exemplary embodiment, the light sources 204 may be a bit recessed with respect to the image sensor 206 to prevent light leaks directly into the image sensor 206.

Furthermore, the disclosure provided herein reduces the cost by providing a flexible PCB imaging and illumination device in a more manufacturable design.

The flexible length of the ribbon cable, whether it is formed as a straight unit, or with the described serpentine pattern, moves the rigid solder connections away from the distal active portion, resulting in a shorter distal portion of the PCB 202 that is rigid due to the presence of components and electrical connections. This will allow the flexible PCB camera 200 to pass through tighter bends when the bendable medical device 13 has been navigated to tortuous anatomy.

In addition, having two arms 212 minimizes the profile of the flexible PCB camera 200 to better fit into the endoscope tool channel 108 and to better traverse the tool channel 108 during reinsertion. With the addition of a third arm 212, a second light source 204 can be incorporated, so the illumination will be more balanced around the single camera 206. Also, a second light source 204 will provide more illumination, resulting in lower input power, which will reduce heat generated by the flexible PCB camera 200. Further use of the third arm to comprise a second camera 206 would allow for 3D imaging.

### Definitions

In referring to the description, specific details are set forth in order to provide

a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. As used herein, the term “substantially” is meant to allow for deviations from the descriptor that do not negatively affect the intended purpose. For example, deviations that are from limitations in measurements, differences within manufacture tolerance, or variations less than 5% can be considered within the scope of substantially the same. The specified descriptor can be absolute value (e.g. substantially spherical, substantially perpendicular, etc.) or relative (e.g. substantially not differing height, substantially the same, etc.).

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A camera module comprising:
a flexible printed circuit board having at least a first arm and a second arm;
an image sensor mounted on the first arm;
a light source mounted on the second arm; and
a housing encompassing the flexible printed circuit board, image sensor and light source,
wherein both the first arm and second arm are manipulated, independent of each other, to align the image sensor with the light source.

2. The camera module of claim 1, wherein an orientation of the aligning of the image sensor with the light source is perpendicular to the length of the flexible printed circuit board.

3. The camera module of claim 1, wherein the aligning of the image sensor with the light source comprises bending each of the arms holding the image sensor and light source by approximately ninety degrees.

4. The camera module of claim 1, wherein the image sensor is a camera or a CMOS sensor.

5. The camera module of claim 1, wherein the aligning of the image sensor with the light source results in a top surface of the image sensor and a top surface of the light source being parallel with each other.

6. The camera module of claim 1, further comprising a third arm configured for manipulation to align with the image sensor and the light source.

7. The camera module of claim 6, wherein a second light source is mounted to the third arm. (this provides more even illumination-potentially could reduce the amount of power provided to each light source; this would also reduce the amount of heat generated).

8. The camera module of claim 1, wherein the second arm is angled towards the center of the camera module (image sensor has conical view; LED is offset slightly, this aims the center of the light emission towards the center of the image sensor field of view).

9. The camera module of claim 1, wherein the image sensor and the light source are mounted on the same side of the flexible printed circuit board.

10. The camera module of claim 1, wherein the first arm or second arm may be folded multiple times.

11. The camera module of claim 1, further comprising a conductor of a coaxial cable soldered to the flexible PCB at the proximal end of the flexible PCB.

12. The camera module of claim 7, wherein the third arm is angled towards the center of the camera module.

13. The camera module of claim 6, wherein the third arm may be folded multiple times.

14. The camera module of claim 1, wherein the flexible PCB is extended to provide a ribbon cable having electrical connections at the proximal end.

15. A method for imaging anatomy of a patient, comprising:
inserting a camera module into a catheter;
inserting the catheter into the anatomy to reach a point of interest; and
capturing one or more images or videos of the point of interest using the camera module:
wherein the camera module comprises:
a flexible printed circuit board having at least a first arm and a second arm;
an image sensor mounted on the first arm;
a light-source mounted on the second arm; and
a housing encompassing the flexible printed circuit board, image sensor and light source,
wherein both the first arm and second arm are manipulated, independent of each other, to align the image sensor with the light source.

16. A printed circuit board having an active portion, a flexible portion and a connection portion, wherein the flexible portion is located between the active portion and the connection portion:
wherein the active portion has electrically attached thereto at least one image sensor,
the flexible portion comprises a flexible PCB material having printed thereon conductors electrically connecting the image sensor in the active portion and the connection portion, and
the connection portion comprises means for electrical connection to a power and/or control source;
wherein no attachment means or soldering is present in the flexible portion.

17. A camera module comprising:
the printed circuit board of claim 16, wherein the active portion comprises:
at least a first arm and a second arm;
the image sensor mounted on the first arm;
a light source mounted on the second arm; and
wherein the flexible portion is configured to extend a distance lengthwise through an endoscope for a distance that is at least five times the diameter of the endoscope.

18. The camera module of claim 17. additionally comprising a cover surrounding the active portion.

19. The printed circuit board of claim 16, wherein the flexible portion comprises a serpentine path that can be unbent to extend to full length.

20. The printed circuit board of claim 16, wherein the flexible portion is comprises a straight length of material.
